(19) United States
(12) Patent Application Publication
(10) Pub. No.: US 2009/0149520 A1
(43) Pub. Date: Jun. 11, 2009

(54) OXAZOLIDINONES CONTAINING OXINDOLES AS ANTIBACTERIAL AGENTS

(75) Inventors: Gary Wayne Luehr, Hayward, CA (US); Sumithra Gowlugari, San Mateo, CA (US); Mikhail Fedorovich Gordeev, Castro Valley, CA (US); Vara Prasad Venkata Nagendra Josyula, Superior Township, MI (US)

Correspondence Address: PFIZER INC;Steve T. Zelson, 150 EAST 42ND STREET, 5TH FLOOR - STOP 49, NEW YORK, NY, 10017-5612, US

(73) Assignee: PFIZER INC

(21) Appl. No.: 12/278,587

(22) PCT Filed: Feb. 6, 2007
(86) PCT No.: PCT/IB2007/000284

§ 371 Date: Aug. 7, 2008

Related U.S. Application Data
(60) Provisional application No. 60/771,570, filed on Feb. 8, 2006.

(51) Int. Cl. A61K 31/422; C07D 263/18; A61P 31/04

(52) U.S. Cl. 514/376; 548/229

(57) ABSTRACT

The present invention provides a compound of formula (I) or a pharmaceutically acceptable salt thereof wherein X is C₁₋₄alkyl; and Y is H or F. The compounds of the present invention are useful as antibacterial agents.

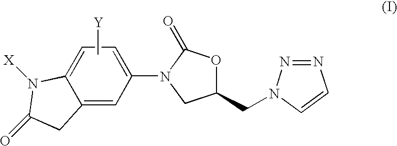

### FIELD OF INVENTION

The present invention relates to novel oxindole derivatives of oxazolidinones, pharmaceutical compositions thereof, methods for their use, and methods for preparation. These compounds have potent activities against gram-positive bacteria.

### BACKGROUND OF THE INVENTION

Antibacterial resistance is a global clinical and public health problem that has emerged with alarming rapidity in recent years and undoubtedly will increase in the near future. Resistance is a problem in the community as well as in health care settings, where transmission of bacteria is greatly amplified. Because multiple drug resistance is a growing problem, physicians are now confronted with infections for which there is no effective therapy. As result, structurally novel antibacterials with a new mode of action have become increasingly important in the treatment of bacterial infections.

Among newer antibacterial agents, oxazolidinone compounds are the most recent synthetic class of antimicrobials active against a number of pathogenic microorganisms. This invention provides novel oxindole derivatives of oxazolidinones, and their preparation.

### INFORMATION DISCLOSURE

PCT publication WO 0181350 discloses oxazolidinone derivatives containg an N-linked heterocycle as antibacterial agents.

PCT publication WO 03072575 discloses oxazolidinone derivatives incorporating a nitrogen containing 5-membered ring and their use as antibacterial agents.

PCT publication WO 2004/074282 discloses antibacterial indolone oxazolidinones.

### SUMMARY OF THE INVENTION

The present invention provides a compound of formula I

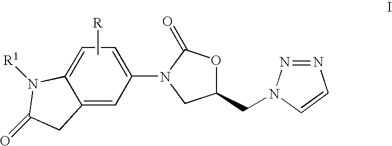

or a pharmaceutically acceptable salt thereof wherein:

R is H or F; and R¹ is C₁₋₄alkyl.

In another aspect, the present invention also provides:

a pharmaceutical composition which comprises a pharmaceutically acceptable carrier and an effective amount of a compound of formula I,

a method for treating gram-positive microbial infections in a mammal by administering to the subject in need a therapeutically effective amount of a compound of formula I or a pharmaceutically acceptable salt thereof, and

a use of a compound of formula I or a pharmaceutically acceptable salt thereof to prepare a medicament for treating gram-positive microbial infections.

The invention may also provide novel intermediates and novel processes that are useful for preparing compounds of formula I.

### DETAILED DESCRIPTION OF THE INVENTION

Unless otherwise stated, the following terms used in the specification and claims have the meanings given below:

The carbon atom content of various hydrocarbon-containing moieties is indicated by a prefix designating the minimum and maximum number of carbon atoms in the moiety, i.e., the prefix Cᵢ₋ⱼ indicates a moiety of the integer “i” to the integer “j” carbon atoms, inclusive. Thus, for example, C₁₋₄ alkyl refers to alkyl of one to four carbon atoms, inclusive.

The term alkyl refers to both straight and branched groups, but reference to an individual radical such as “propyl” embraces only the straight chain radical, a branched chain isomer such as “isopropyl” being specifically referred to.

The term “a pharmaceutically acceptable salt” of a compound means a salt that is pharmaceutically acceptable and that possesses the desired pharmacological activity of the parent compound. Such salts include:

(1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or

(2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

The term “pharmaceutically acceptable carrier” means a carrier that is useful in preparing a pharmaceutical composition that is generally safe, non-toxic and neither biologically nor otherwise undesirable, and includes a carrier that is acceptable for veterinary use as well as human pharmaceutical use. “A pharmaceutically acceptable carrier” as used in the specification and claims includes both one and more than one such carrier.

The term “mammal” refers to human or warm-blooded animals including livestock and companion animals.

The term “optional” or “optionally” means that the subsequently described event or circumstance may, but need not, occur, and that the description includes instances where the event or circumstance occurs and instances in which it does not.

Compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this invention may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 1992).

The term “treating” or “treatment” of a disease includes: (1) preventing the disease, i.e. causing the clinical symptoms of the disease not to develop in a mammal that may be exposed to or predisposed to the disease but does not yet experience or display symptoms of the disease; (2) inhibiting the disease, i.e., arresting or reducing the development of the disease or its clinical symptoms; or (3) relieving the disease, i.e., causing regression of the disease or its clinical symptoms.

The term “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

The term “leaving group” has the meaning conventionally associated with it in synthetic organic chemistry i.e., an atom or group capable of being displaced by a nucleophile and includes halogen, alkylsulfonyloxy, ester, or amino such as chloro, bromo, iodo, mesyloxy, tosyloxy, trifluorosulfonyloxy, methoxy, N,O-dimethylhydroxyl-amino, and the like.

The compounds of the present invention are generally named according to the IUPAC or CAS nomenclature system.

Abbreviations which are well known to one of ordinary skill in the art may be used (e.g. “Ph” for phenyl, “Me” for methyl, “Et” for ethyl, “h” for an hour or hours and “rt” for room temperature).

Specific and preferred values listed below for radicals, substituents, and ranges, are for illustration only; they do not exclude other defined values or other values within defined ranges for the radicals and substituents.

Specifically, X is methyl, ethyl, propyl, isopropyl, butyl, iso-butyl, sec-butyl, and their isomeric forms thereof.

Specifically Y is H.

Examples of the present invention include:
(1)-5-(1H-1,2,3-triazol-1-yl)methyl)-3-(1-isopropyl-2-oxoindolin-5-yl)oxazolidin-2-one,
(2)-5-(1H-1,2,3-triazol-1-yl)methyl)-3-(1-ethyl-2-oxoindolin-5-yl)oxazolidin-2-one, or
(3)-5-(1H-1,2,3-triazol-1-yl)methyl)-3-(1-methyl-2-oxoindolin-5-yl)oxazolidin-2-one.

Compounds of this invention can be prepared in accordance with one or more of the Schemes discussed below. All of the starting materials are either commercially available or can be prepared by procedures that would be well known to one of ordinary skill in organic chemistry. The variables used in the Schemes are as defined below, or as in the summary of the invention or claims.

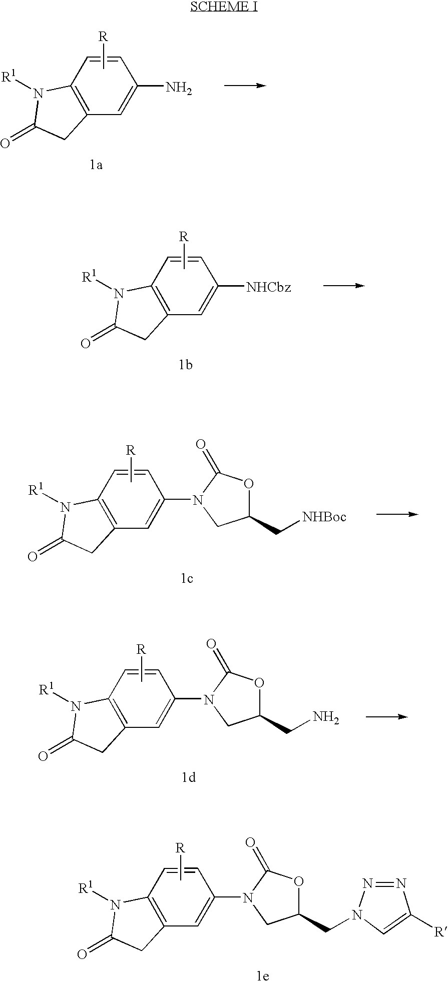

Scheme I illustrates the construction of an oxazolidinone ring bearing a 1,2,3-triazole-1-yl methyl group at the C-5 position. The aniline intermediate 1a is first converted to an aryl carbamate using standard procedures that are well known to those skilled in the art, for example using benzyl chloroformate and pyridine in dichloromethane. The aryl carbamate 1b is reacted with (S)-(3-chloro-2-hydroxy-propyl)-carbamic acid tert-butyl ester (prepared as described in U.S. Pat. No. 6,833,453). The reaction is performed in the presence of an organic base such as lithium tert-butoxide, in a polar organic solvent such as dimethylformamide or acetonitrile, at temperatures of about 0° C. to 25° C. The product may be used as collected or may first be purifed using conventional techniques such as preparative TLC or HPLC, chromatography, precipitation, crystallization and the like. The product of this reaction is intermediate 1c bearing a tert-butyl carbamate (Boc protected amine) at the C-5 position. Intermediate 1c is deprotected by treatment with acids such as hydrochloric acid or trifluoracetic acid. If less harsh conditions are required, treatment with trimethylsilyltrifluoromethane sulfonate and 2,6-lutidine (as described by Ohfune, Y. and Sakaitani, M. J. Org. Chem. 1990, 55, 870-876) is also effective. Finally, amine 1d is reacted with an appropriately substituted arenesulfonyl hydrazone according to the methods of Ichikawa (Chem. Pharm. Bull., 2000, 48, 1947-1953) and Sakai (Bull. Chem. Soc. Jpn., 1986, 59, 179-184) to provide triazole oxazolidinone 1e werein R′ is hydrogen or other substituents such as halo, alkyl, and etc.

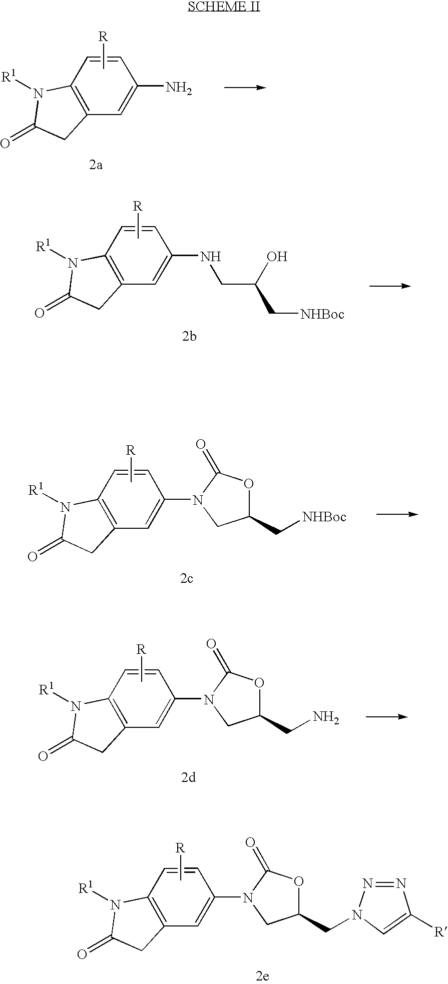

Alternatively as described in Scheme II, 2a is reacted with a Boc protected (S)-glycidylamine (prepared following methods described in International Patent Publication No. WO 02/32857) in the presence of a Lewis acid such as lithium triflate in a suitable solvent such as acetonitrile at a suitable temperature, typically in the range from 20° C. to 110° C. to provide 2b. The amino alcohol 2b can then be ring closed to give the aryl oxazolidinones 2c using methods known to one skilled in the art. For instance, treatment of structures 2b with 1,1′-carbonyldiimidazole in a solvent such as acetonitrile or tetrahydrofuran at an appropriate temperature, typically in a range of 20° C. to 60° C., or with phosgene in a solvent such as toluene or methylene chloride, or mixtures thereof, in the presence of a base such as triethylamine at an appropriate temperature, typically in a range from −10° C. to 25° C., affords 2c. Oxazolidinone 2c may be deprotected and converted to triazole 2e following methods described in Scheme I.

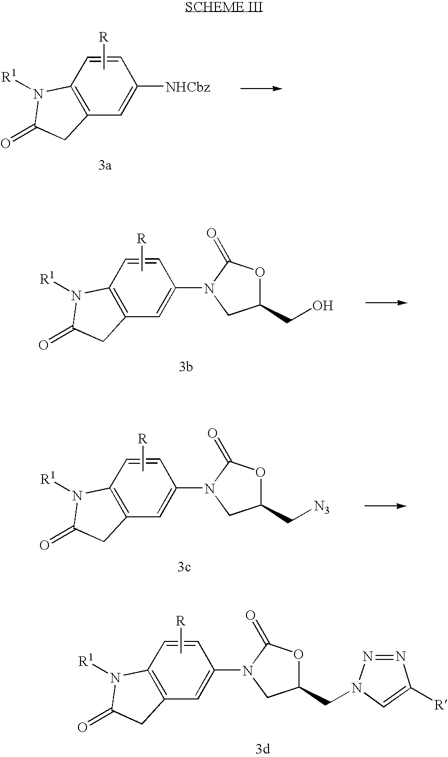

In another embodiment of the reaction process described in Scheme III, carbamate 3a is converted to the azido oxazolidinone 3c following the sequence of chemical transformations described by Brickner (J. Med. Chem., 1996, 39, 673-679). Cycloaddition of the intermediate azido compound with norbornadiene in a suitable solvent, such as dioxane at reaction temperatures in the range of about 50° C. to about 100° C. affords the 1,2,3-triazolyl derivative 3d (R′=H). Alternatively, a variety of other substituted triazoles (R′=Me, Cl, F, —OH, —CH₂OH, —CH₂CN, —CN, —C═CH, —NH₂) may be prepared via cycloaddition in the presence of Cu(I) catalysis as described by Rostovtsec (Angew. Chem. Int. Ed., 2002, 41, 2596-2599) and subsequent chemical group modification by known methods when necessary.

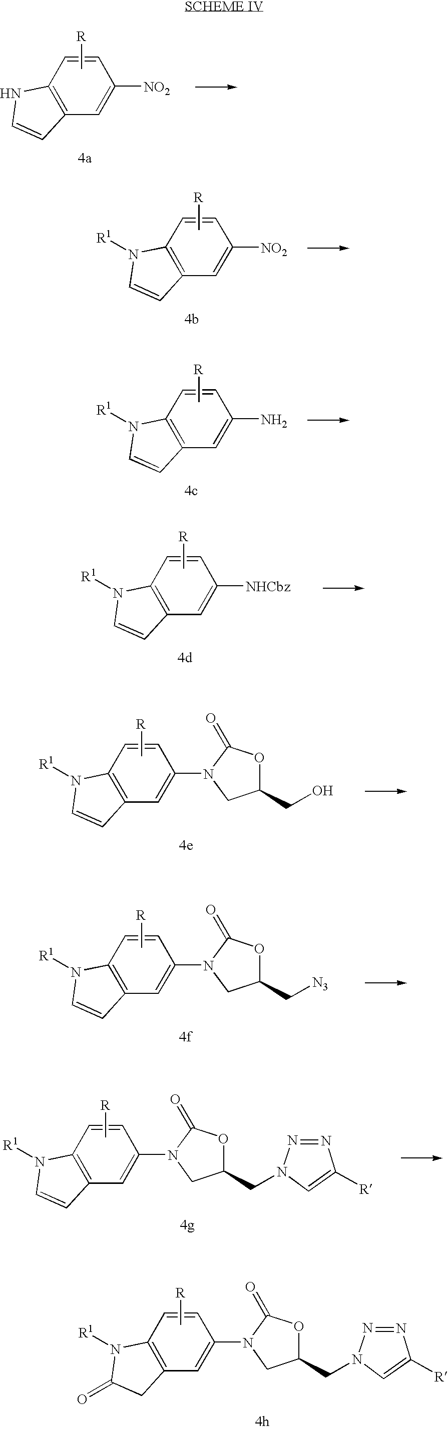

Scheme IV exemplifies another route to prepare oxazolidinones 4e via oxidation of indole precursors. Appropriately substituted 5-nitroindole 4a is reacted with an appropriate alkylating agent such as an alkyl halide, tosylate or sulfate in the presence of a strong base such as sodium hydride in a polar aprotic solvent such as dimethylformamide to provide N-alkylindole 4b. 4b is reduced under a variety of known conditions including catalytic hydrogenation over a noble metal catalyst or a Raney Nickel catalyst, a dissolving metal reduction such as iron and ammonium chloride in aqueous ethanol or iron in acetic acid to provide aniline 4c. 4c is converted to the aryl carmbamate 4d, the azido compound 4f, and the triazole oxazolidinone 4g following the sequence of chemical transformations described in the previous scheme. N-Alkylindole 4g is further oxidized to the requisite oxindole 4h by a variety of known methods (e.g. DMSO/HCl, NBS).

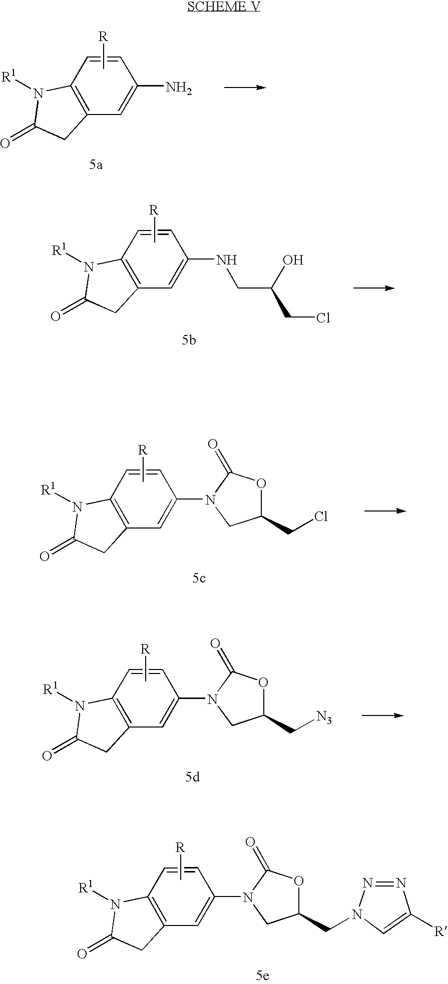

Triazole oxazolidinones 5e may also be prepared following the route detailed in Scheme V. Oxindole amine 5a is reacted with the conmmercially available (R)-(−)-epichlorohydrin either neat or in a suitable inert solvent such as acetonitrile or isopropanol at temperatures between ambient to about 100° C. to afford 5b. A Lewis acid catalyst such as lithium triflate may be added. Aminoalcohol 5b can then be ring closed to give the aryl oxazolidinones 5c using methods known to one skilled in the art. For instance, treatment of structures 5b with 1,1′-carbonyldiimidazole in a solvent such as acetonitrile or tetrahydrofuran at an appropriate temperature, typically in a range of 20° C. to 60° C., or with phosgene in a solvent such as toluene or methylene chloride, or mixtures thereof, in the presence of a base such as triethylamine at an appropriate temperature, typically in a range from −10° C. to 25° C., affords 5c. Reaction of the chloro oxazolidinone 5c with an azide source such as sodium azide in a suitable solvent such as dimethylformamide at temperatures between ambient to about 75° C. provides 5d. Azido oxazolidinone 5d may be converted to triazole oxazolidinones 5e by methods described previously in Scheme III.

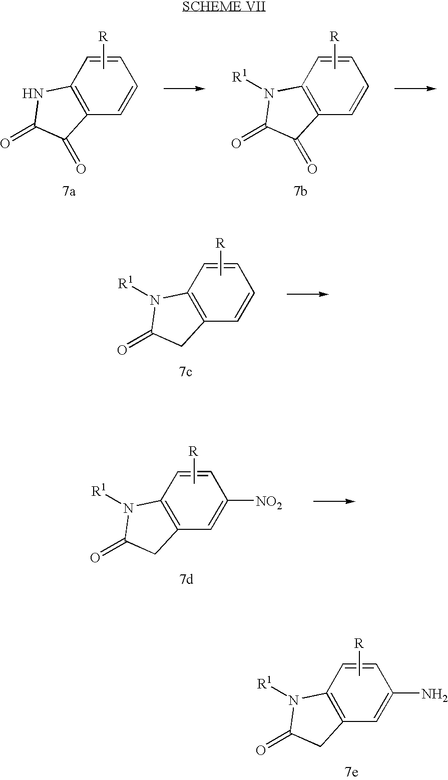

Oxindole intermediates may be prepared according to the method of Scheme VII. Isatin 7a, obtained commercially or conveniently prepared according to the methods of Gassman described in J. Org. Chem. 1977, 42, 1344 and U.S. Pat. Nos. 4,188,325 and 4,2527,23, is treated with an alkylating agent, e.g., iodomethane, iodoethane, or iodopropane, in the presence of a suitable base (e.g. an amine base such as triethylamine or di-iso-propylethylamine or lithium, sodium, potassium or cesium carbonate) in a suitable organic solvent (e.g. DMF, THF, DMSO, dioxane or acetonitrile) at a temperature between 0° C. and 65° C. to afford N-alkylated isatin 7b. Isatins 7b may be reduced to 1,3-dihydroindol-2-ones 7c by using red phosphorous and iodic acid, by use of hydrogen sulfide in pyridine/co-solvent mixture, or by the Wolf-Kishner reaction. The most convenient procedure involves heating isatin 7b in neat hydrazine hydrate at reflux in the absence of any additional base. 1,3-Dihydroindol-2-one 7c is nitrated regioselectively using methods known to one skilled in the art (e.g., nitric acid in concentrated sulfuric acid or acetic acid, or sodium nitrate in trifluoroacetic acid at temperatures between −20° C. and 25° C.). 5-Nitrooxindole 7d is then reduced by dissolving metal reduction (e.g., iron and ammonium chloride in ethanol/water) or catalytic hydrogenation to provide the 5-aminooxindole 7e.

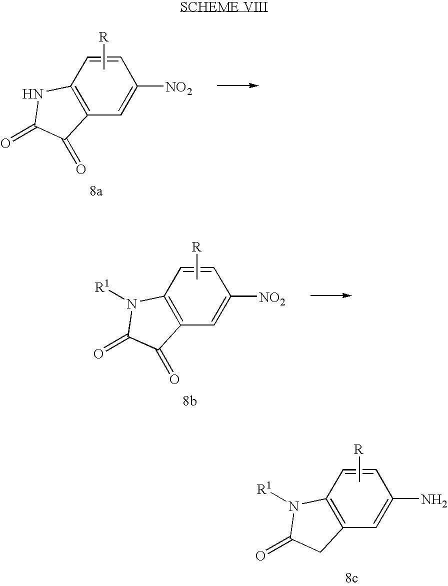

Alternatively, known 5-nitroisatins 8a are treated with an appropriate alkylating agent, e.g., iodomethane, iodoethane, or iodopropane, in the presence of a suitable base base (e.g. an amine base such as triethylamine or di-iso-propylethylamine or lithium, sodium, potassium or cesium carbonate) in a suitable organic solvent (e.g. DMF, TF, DMSO, dioxane or acetonitrile) at a temperature between 0° C. and 65° C. to afford N-alkylated isatin 8b. Isatin 8b may be reduced in one step to the requisite 5-aminooxindole 8c by heating in neat hydrazine hydrate at reflux temperatures or by catalytic hydrogenation.

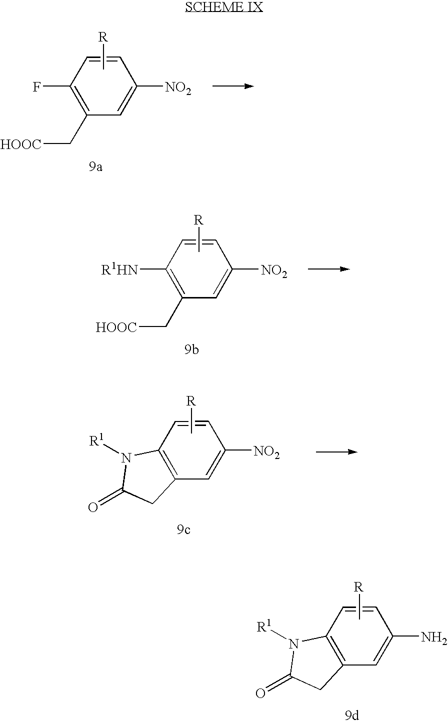

In another route exemplified by Scheme IX, an appropriately substituted 2-halo-5-nitrophenylacetic acid 9a (e.g., preferably 2-fluoro-5-nitrophenylacetic acid) is treated with ammonia or an optionally substituted amine (R¹NH₂) in a suitable solvent such as DMSO or acetonitrile at temperatures between 35° C. and 85° C. to afford 9b (R¹=H or optionally substituted alkyl). Aniline 9b is treated with a strong acid such as HCl, H₂SO₄, or TFA to effect cyclization to the requisite 5-nitrooxindole 9c. 5-Nitrooxindole 9c is then reduced by dissolving metal reduction (e.g., iron and ammonium chloride in ethanoVwater) or catalytic hydrogenation to provide the 5-aminooxindole 9d.

### Medical and Veterinary Uses

The compound of the present invention may be used for the treatment of infectious, Gram-positive bacterial infections caused by a variety of bacterial organisms, including those that require long-term therapy (>28 days).

Examples of the bacterial organisms include gram-positive bacteria such as multiple resistant staphylococci, for example S. aureus and S. epidermidis; multiple resistant streptococci, for example S. pneumoniae and S. pyogenes; and multiple resistant Enterococci, for example E. faecalis; gram negative aerobic bacteria such as Haemophilus, for example H. influenzae and Moraxella, for example M. catarrhalis; as well as anaerobic organisms such as bacteroides and clostridia species, and acid-fast organisms such as Mycobacteria, for example M. tuberculosis; and/or Mycobacterium avium. Other examples include Escherichia, for example E. coli. intercellular microbes, for example Chlamydia and Rickettsiae.

Examples of infections that may be treated with the compound of the present invention include central nervous system infections, external ear infections, infections of the middle ear, such as acute otitis media, infections of the cranial sinuses, eye infections, infections of the oral cavity, such as infections of the teeth, gums and mucosa, upper respiratory tract infections, lower respiratory tract infections, genitourinary infections, gastrointestinal infections, gynecological infections, septicemia, bone and joint infections, skin and skin structure infections, bacterial endocarditis, burns, antibacterial prophylaxis of surgery, and antibacterial prophylaxis in immunosuppressed patients, such as patients receiving cancer chemotherapy, or organ transplant patients. Specifically, infectious diseases that may be treated with the compound of the present invention are gram-positive infections such as osteomyelitis, endocarditis and diabetic foot.

### Antibacterial Activity

The in vitro antibacterial activity of the compounds of the present invention may be assessed by following procedures recommended in (1) National Committee for Clinical Laboratory Standards (January 2003), Methods for dilution antimicrobial tests for bacteria that grow aerobically, Approved Standard (6^{th} ed), M7-A6, NCCLS, Wayne, Pa.; (2) National Committee for Clinical Laboratory Standards (March 2001), Methods for antimicrobial susceptibility testing of anaerobic bacteria, Approved Standard (5^{th} ed), M11-A4, NCCLS, Wayne, Pa.; (3) National Committee for Clinical Laboratory Standards (Jan. 2003), MIC testing supplemental tables, M100-S13 (for use with M7-A6), NCCLS, Wayne, Pa.; and (4) Murray P R, Baron E J, Jorgensen J H, et al. Manual of Clinical Microbiology (8^{th} ed) Washington, D.C.: American Society for Microbiology Press, 2003. The antibacterial activity can be presented in the form of MIC value. The MIC value is the lowest concentration of drug, which prevented macroscopically visible growth under the conditions of the test. Table 1 lists the in vitro antibacterial activity of the present invention.

TABLE 1
Results of in vitro antibacterial activity MICₛ (μg/mL)

| | S. aureus | S. pneumoniae | E. faecalis |
|---|---|---|---|
| Example No. | UC-76 SA-1 | SV1 SP-3 | MGH-2 EF 1-1 |
| 1 | 8 | 8 | 8 |
| 2 | 4 | 4 | 8 |

### Pharmaceutical Salts

The compound of formula I may be used in its native form or as a salt. In cases where forming a stable nontoxic acid or base salt is desired, administration of the compound as a pharmaceutically acceptable salt may be appropriate. Examples of pharmaceutically acceptable salts of the present invention include inorganic salts such as hydrochloride, hydrobromide, sulfate, nitrate, bicarbonate, carbonate salts, and organic salts such as tosylate, methanesulfonate, acetate, citrate, malonate, tartarate, succinate, benzoate, ascorbate, etoglutarate, and glycerophosphate. Pharmaceutically acceptable salts may be obtained using standard procedures well known in the art, for example, reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

Routes of Administration

In therapeutic use for treating, or combating, bacterial infections in a mammal (i.e. human and animals), a compound of the present invention or its pharmaceutical compositions can be administered orally, parenterally, topically, rectally, transmucosally, or intestinally.

Parenteral administrations include indirect injections to generate a systemic effect or direct injections to the afflicted area. Examples of parenteral administrations are subcutaneous, intravenous, intramuscular, intradermal, intrathecal, intraocular, intranasal, intravetricular injections or infusions techniques.

Topical administrations include the treatment of infectious areas or organs readily accessibly by local application, such as, for example, eyes, ears including external and middle ear infections, vaginal, open wound, skins including the surface skin and the underneath dermal structures, or other lower intestinal tract. It also includes transdermal delivery to generate a systemic effect.

The rectal administration includes the form of suppositories.

The transmucosal administration includes nasal aerosol or inhalation applications.

The preferred routes of administration are oral and parenteral.

### Composition/Formulation

Pharmaceutical compositions of the present invention may be manufactured by processes well known in the art, e.g., by means of conventional mixing, dissolving, granulation, dragee-making, levigating, emulsifying, encapsulating, entrapping, lyophilizing processes or spray drying.

Pharmaceutical compositions for use in accordance with the present invention may be formulated in conventional manner using one or more physiologically acceptable carriers comprising excipients and auxiliaries, which facilitate processing of the active compound into preparations, which can be used pharmaceutically. Proper formulation is dependent upon the route of administration chosen.

For oral administration, the compound can be formulated by combining the active compound with pharmaceutically acceptable carriers well known in the art. Such carriers enable the compound of the invention to be formulated as tablets, pills, lozenges, dragees, capsules, liquids, solutions, emulsions, gels, syrups, slurries, suspensions and the like, for oral ingestion by a patient. A carrier can be at least one substance which may also function as a diluent, flavoring agent, solubilizer, lubricant, suspending agent, binder, tablet disintegrating agent, and encapsulating agent. Examples of such carriers or excipients include, but are not limited to, magnesium carbonate, magnesium stearate, talc, sugar, lactose, sucrose, pectin, dextrin, mannitol, sorbitol, starches, gelatin, cellulosic materials, low melting wax, cocoa butter or powder, polymers such as polyethylene glycols and other pharmaceutical acceptable materials.

Dragee cores are provided with suitable coatings. For this purpose, concentrated sugar solutions may be used which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions, which can be used orally, include push-fit capsules made of gelatin, as well as soft, sealed capsules made of gelatin and a plasticizer, such as glycerol or sorbitol. The push-fit capsules can contain the active ingredients in admixture with a filler such as lactose, a binder such as starch, and/or a lubricant such as talc or magnesium stearate and, optionally, stabilizers. In soft capsules, the active compound may be dissolved or suspended in suitable liquids, such as fatty oils, liquid paraffin, liquid polyethylene glycols, cremophor, capmul, medium or long chain mono-, di- or triglycerides. Stabilizers may be added in these formulations, also.

Liquid form compositions include solutions, suspensions and emulsions. For example, there may be provided solutions of the compound of this invention dissolved in water and water-propylene glycol and water-polyethylene glycol systems, optionally containing suitable conventional coloring agents, flavoring agents, stabilizers and thickening agents.

The compound may also be formulated for parenteral administration, e.g., by injections, bolus injection or continuous infusion. Formulations for parenteral administration may be presented in unit dosage form, e.g., in ampoules or in multi-dose containers, with an added preservative. The compositions may take such forms as suspensions, solutions or emulsions in oily or aqueous vehicles, and may contain formulating materials such as suspending, stabilizing and/or dispersing agents.

For injection, the compound of the invention may be formulated in aqueous solution, preferably in physiologically compatible buffers or physiological saline buffer. Suitable buffering agents include tri-sodium orthophosphate, sodium bicarbonate, sodium citrate, N-methylglucamine, L(+)-lysine and L(+)-arginine.

Parenteral administrations also include aqueous solutions of a water soluble form, such as, without limitation, a salt, of the active compound. Additionally, suspensions of the active compound may be prepared in a lipophilic vehicle. Suitable lipophilic vehicles include fatty oils such as sesame oil, synthetic fatty acid esters such as ethyl oleate and triglycerides, or materials such as liposomes. Aqueous injection suspensions may contain substances, which increase the viscosity of the suspension, such as sodium carboxymethyl cellulose, sorbitol, or dextran. Optionally, the suspension may also contain suitable stabilizers and/or agents that increase the solubility of the compound to allow for the preparation of highly concentrated solutions.

Alternatively, the active ingredient may be in powder form for constitution with a suitable vehicle, e.g., sterile, pyrogen-free water, before use.

For suppository administration, the compound may also be formulated by mixing the agent with a suitable non-irritating excipient, which is solid at room temperature but liquid at rectal temperature and therefore will melt in the rectum to release the drug. Such materials include cocoa butter, beeswax and other glycerides.

For administration by inhalation, compound of the present invention can be conveniently delivered through an aerosol spray in the form of solution, dry powder, or suspensions. The aerosol may use a pressurized pack or a nebulizer and a suitable propellant. In the case of a pressurized aerosol, the dosage unit may be controlled by providing a valve to deliver a metered amount. Capsules and cartridges of, for example, gelatin for use in an inhaler may be formulated containing a power base such as lactose or starch.

For topical applications, the pharmaceutical composition may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion such as suspensions, emulsion, or cream containing the active components suspended or dissolved in one or more pharmaceutically acceptable carriers. Suitable carriers include, but are not limited to, mineral oil, sorbitan monosterate, polysorbate 60, cetyl esters wax, ceteary alcohol, 2-octyldodecanol, benzyl alcohol and water.

For ophthalmic and otitis uses, the pharmaceutical compositions may be formulated as micronized suspensions in isotonic, pH adjusted sterile saline, or preferably, as solutions in isotonic, pH adjusted sterile saline, either with or without a preservative such as a benzylalkonium chloride. Alternatively, for ophthalmic uses, the pharmaceutical compositions may be formulated in an ointment such as petrolatum.

In addition to the formulations described previously, the compound may also be formulated as depot preparations. Such long acting formulations may be in the form of implants. A compound of this invention may be formulated for this route of administration with suitable polymers, hydrophobic materials, or as a sparing soluble derivative such as, without limitation, a sparingly soluble salt.

Additionally, the compound may be delivered using a sustained-release system. Various sustained-release materials have been established and are well known by those skilled in the art. Sustained-release capsules may, depending on their chemical nature, release the compound for 24 hours or for up to several days.

### Dosage

Pharmaceutical compositions suitable for use in the present invention include compositions wherein the active ingredients are contained in an amount sufficient to achieve the intended purpose, i.e., the treatment or prevent of infectious diseases. More specifically, a therapeutically effective amount means an amount of compound effective to prevent, alleviate or ameliorate symptoms of disease or prolong the survival of the subject being treated.

The quantity of active component, that is the compound of this invention, in the pharmaceutical composition and unit dosage form thereof may be varied or adjusted widely depending upon the manner of administration, the potency of the particular compound and the desired concentration. Determination of a therapeutically effective amount is well within the capability of those skilled in the art. Generally, the quantity of active component will range between 0.5% to 90% by weight of the composition.

Generally, a therapeutically effective amount of dosage of active component will be in the range of about 0.1 to about 400 mg/kg of body weight/day, more preferably about 1.0 to about 50 mg/kg of body weight/day. It is to be understood that the dosages may vary depending upon the requirements of each subject and the severity of the bacterial infection being treated. In average, the effective amount of active component is about 200 mg to 800 mg and preferable 600 mg per day.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

Also, it is to be understood that the initial dosage administered may be increased beyond the above upper level in order to rapidly achieve the desired plasma concentration. On the other hand, the initial dosage may be smaller than the optimum and the daily dosage may be progressively increased during the course of treatment depending on the particular situation. If desired, the daily dose may also be divided into multiple doses for administration, e.g., two to four times per day.

In cases of local administration or selective uptake, the effective local concentration of the drug may not be related to plasma concentration and other procedures know in the art may be used to determine the desired dosage amount.

### EXAMPLES

In the discussion above and in the examples below, the following abbreviations have the following meanings. If an abbreviation is not defined, it has its generally accepted meaning.
bm=broad multiplet
BOC=tert-butoxycarbonyl
bd=broad doublet
bs=broad singlet
CDI=1,1O-carbodiimidazole
d=doublet
dd=doublet of doublets
dq=doublet of quartets
dt=doublet of triplets
DMF=dimethylformamide
DMAP=dimethylaminopyridine
DMSO=dimethyl sulfoxide
eq.=equivalents
g=grams
h=hours
HPLC=high pressure liquid chromatography
HATU=N-[(dimethylamino)-1H-1,2,3-triazolo-[4,5-b]pyridin-1-yl-methylene]-N-methylmethanaminium hexafluorophosphate N-oxide
LG leaving group
m=multiplet
M=molar
M %=mole percent
max=maximum
meq=milliequivalent
mg=milligram
mL=milliliter
mm=millimeter
mmol=millimol
q=quartet
s=singlet
t or tr=triplet
TBS=tributylsilyl
TFA=trifluoroacetic acid
THF=tetrahydrofuran
TLC thin layer chromatography
p-TLC=preparative thin layer chromatography
μL=microliter
N=normality
MeOH=methanol
DCM=dichloromethane
HCl=hydrochloric acid
ACN=acetonitrile
MS=mass spectrometry
rt=room temperature
EtOAc=ethyl acetate
EtO=ethoxy
Ac=acetate
NMP=1-methyl-2-pyrrolidinone
μL=microliter
J=coupling constant
NMR=Nuclear magnetic resonance
MHz=megahertz
Hz=hertz
m/z=mass to charge ratio
min=minutes
Boc=tert-butoxycarbonyl
CBZ=benzyloxycarbonyl
DCC=1,3-dicyclohexylcarbodimide
PyBop=benzotriazole-1-yl-oxy-trispyrrolidinophosphonium hexafluorophosphate

### Example 1

### Preparation of (R)-5-((1H-1,2,3-triazol-1-yl)methyl)-3-(1-isopropyl-2 oxoindolin-5-yl)oxazolidin-2-one

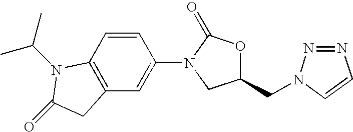

### Method A

Step 1. Preparation of 1-isopropyl-5-nitro-1H-indole.

5-Nitro-1H-indole (2.00 g, 0.012 mol) in dimethylformamide (8 mL) is added to a suspension of sodium hydride (60% in mineral oil, 0.71 g, 0.015 mol) in dimethylformamide (30 mL) at 0° C. After 30 min at 0° C., isopropyl iodide (1.35 mL, 0.013 mol) is added and the mixture stirred for 6 h at room temperature. The reaction is diluted with water and extracted with ethyl acetate. The organic layer is separated, washed with water and brine, dried (Na₂SO₄) and evaporated. The residue is purified by flash column chromatography to provide the title compound. HPLC(SYMMETRY C₁₈ 3.5 μM, 4.6×30 mm column; gradient elution 2%-98% MeCN with 0.1% TFA over 10 min; 2 mL/min rate): retention time=5.862 min; MS for C₁₁H₁₂N₂O₂ (m/z) 203.0 (M−H)⁺.

Step 2. Preparation of 1-isopropyl-1H-indol-5-amine.

Iron powder (0.549 g, 0.01 mol) is added portionwise to 1-isopropyl-5-nitro-1H-indole (0.5 g, 0.0024 mol) and ammonium chloride (1.309 g, 0.0025 mol) in ethanol (40 mL) and water (20 mL) at 90° C. The mixture is stirred vigorously and heated for 1 hour, allowed to cool, and diluted with dichloromethane (500 mL). The mixture is filtered through celite, washed with water and brine, dried (Na₂SO₄) and evaporated to the title compound. HPLC(SYMMETRY C₁₈ 3.5 μM, 4.6×30 mm column; gradient elution 2%-98% MeCN with 0.1% TFA over 10 min; 2 mL/min rate): retention time=3.208 min; MS for C₁₁H₁₄N₂ ((m/z) 174.2 (M+H)⁺.

Step 3. Preparation of benzyl 1-isopropyl-1H-indol-5-ylcarbamate.

Benzyl chloroformate (0.670 mL, 0.0046 mol) is added dropwise to a mixture 1-isopropyl-1H-indol-5-amine (0.68 g, 0.0039 mol) and pyridine (0.76 mL, 0.008 mol) in dichloromethane (20 mL) at 0° C. The mixture is stirred at 0° C. for 30 min, allowed to warm to room temperature and then diluted with water. The organic layer is separated, washed with brine, dried (Na₂SO₄) and evaporated to provide the title compound. HPLC(SYMMETRY C₁₈ 3.5 μM, 4.6×30 mm column; gradient elution 2%-98% MeCN with 0.1% TFA over 10 min; 2 mL/min rate): retention time=5.834 min; MS for C₁₉H₂₀N₂O₂ (m/z) 309.2 (M+H)⁺.

Step 4. Preparation of (R)-5-(hydroxymethyl)-3-(1-isopropyl-1H-indol-5-yl)oxazolidin-2-one.

Lithium bis(trimethylsilyl)amide (1M in THF, 35.7 mL, 0.036 mol) is added dropwise at −78° C. to benzyl 1-isopropyl-1H-indol-5-ylcarbamate (5.5 g, 0.018 mol) in tetrahydrofuran and the mixture stirred at that temperature for 30 minutes. R-Glycidyl butyrate (2.78 mL, 0.02 mol) is added and the reaction allowed to warm to room temperature and stirred for 14 h. The reaction is quenched with saturated aqueous ammonium chloride, diluted with water and extracted with dichloromethane. The organic layer is washed with brine, dried (Na₂SO₄) and evaporated. The residue is purified by flash column chromatography (20% ethyl acetate/hexane) to provide the title compound. HPLC(SYMMETRY C₁₈ 3.5 μM, 4.6×30 mm column; gradient elution 2%-98% MeCN with 0.1% TFA over 10 min; 2 mL/min rate): retention time=3.153 min; MS for C₁₅H₁₈N₂O₃ (m/z) 275.3 (M+H)⁺.

Step 5. Preparation of (R)-(3-(1-isopropyl-1H-indol-5-yl)-2-oxooxazolidin-5-yl)methyl Methanesulfonate.

Methanesulfonyl chloride (0.84 g, 0.0073 mol) is added at 0° C. to (R)-5-hydroxymethyl)-3-(1-isopropyl-1H-indol-5-yl)oxazolidin-2-one (2.0 g, 0.0073 mol) and triethylamine (1.52 mL, 0.011 mol) in dichloromethane (25 mL) and stirred for 45 minutes. The reaction is quenched with saturated sodium bicarbonate and extracted with dichloromethane. The organic layer is washed with brine, dried (Na₂SO₄) and evaporated to provide the title compound suitable for use directly in the next step. HPLC (SYMMETRY C₁₈ 3.5 μM, 4.6×30 mm column; gradient elution 2%-98% MECN with 0.1% TFA over 10 min; 2 mL/min rate): retention time=5.04 min.

Step 6. Preparation of (R)-5-azidomethyl)-3-(1-isopropyl-1H-indol-5-yl)oxazolidin-2-one.

(R)-3-(1-Isopropyl-1H-indol-5-yl)-2-oxooxazolidin-5-yl)methyl methanesulfonate (3.2 g, 0.0098 mol) and sodium azide (2.372 g, 0.036 mol) in dimethylformamide (15 mL) are heated at 70° C. for 16 h. The reaction is diluted with water and extracted with dichloromethane. The organic layer is washed with brine, dried (Na₂SO₄) and evaporated. The residue is purified by flash column chromatography (20% ethyl acetate/hexane) to provide the title compound. HPLC(SYMMETRY C₁₈ 3.5 μM, 4.6×30 mm column; gradient elution 2%-98% MeCN with 0.1% TFA over 10 min; 2 mL/min rate): retention time=5.429 min; MS for C₁₅H₁₇N₅O₂ (m/z) 300.1 (M+H)⁺.

Step 7. Preparation of (R)-5-((1H-1,2,3-triazol-1-yl)methyl)-3-(1-isopropyl-1H-indol-5-yl)oxazolidin-2-one.

Norbornadiene (1.948 mL, 0.018 mol) and (R)-5-(azidomethyl)-3-(1-isopropyl-1H-indol-5-yl)oxazolidin-2-one (2.7 g, 0.009 mol) in dioxane (20 mL) are heated at 70° C. for 14 h. The reaction is evaporated and the residue purified by flash column chromatography (20% ethyl acetate/hexane) to provide the title compound. HPLC(SYMMETRY C₁₈ 3.5 μM, 4.6×30 mm column; gradient elution 2%-98% MeCN with 0.1% TFA over 10 min; 2 mL/min rate): retention time=4.556 min; MS for C₁₇H₁₉N₅O₂ (m/z) 326.1 (M+H)⁺.

Step 8. Preparation of (R)-5-((1H-1,2,3-triazol-1-yl)methyl)-3-(1-isopropyl-2-oxoindolin-5-yl)oxazolidin-2-one.

N-Bromosuccinimde (0.18 g, 0.001 mol) is added portion wise to (R)-5-((1H-1,2,3-triazol-1-yl)methyl)-3-(1-isopropyl-1H-indol-5-yl)oxazolidin-2-one (0.32 g, 0.001 mol) in a 95:5 mixture of t-butanol and water (3 mL) and stirred for 24 h. The reaction is filtered and the solvent removed under reduced pressure. The residue is diluted with water and extracted with dichloromethane. The organic layer is washed with brine, dried (Na₂SO₄) and evaporated. The residue is purified by PTLC (10% MeOH/DCM) to provide the title compound. HPLC(SYMMETRY C₁₈ 3.5 μM, 4.6×30 mm column; gradient elution 2%-98% MeCN with 0.1% TFA over 10 min; 2 mL/min rate): retention time=3.712 min; MS for C₁₇H₁₉N₅O₃ (m/z) 342.1 (M+H)⁺.

### Method B

Step 1. Preparation of 1-isopropyl-1H-indole-2,3-dione.

1H-Indole-2,3-dione (5.0 g, 0.034 mol), iodopropane (6.83 ml, 0.068 mol) and potassium carbonate (9.28 g, 0.068 mol) in DMF (30 ml) are stirred at room temperature for 72 hours. The reaction mixture is diluted with ethyl acetate, washed with water and brine, dried (Na₂SO₄) and evaporated to provide the title compound. HPLC r.t. 4.38 min; MS for C₁₁H₁₁NO₂M/z 190.1 (M+H)⁺.

Step 2. Preparation of 1-isopropyl-1,3-dihydro-indol-2-one.

1-Isopropyl-1H-indole-2,3-dione (3.00 g, 15.9 mmol) was heated with neat hydrazine hydrate (10 ml) at 130° C. for 1.5 hours. The reaction was cooled, diluted with ice water, and extracted with ethyl acetate. The organic layer is washed with brine, dried (Na₂SO₄), and evaporated to provide the title compound. HPLC r.t. 4.54 min; MS for C₁₁H₁₃NO m/z 176.1 (M+H)⁺.

Step 3. Preparation of 1-isopropyl-5-nitro-1,3-dihydro-indol-2-one.

1-Isopropyl-1,3-dihydro-indol-2-one (2.50 g, 14.3 mmol) is added to a stirred solution of sodium nitrate (1.20 g, 14.26 mmol) in trifluoroacetic acid (50 ml) and stirred at room temperature for 5 h. The reaction was diluted with ice water and resulting precipitate filtered, washed with water, and dried under vacuum to provide the title compound. HPLC r.t. 4.71 min; MS for C₁₁H₁₂N₂O₃ m/z 219.0 (M−H)⁻.

Step 4. Preparation of 5-amino-1-isopropyl-1,3-dihydro-indol-2-one.

Iron powder (2.63 g, 47.2 mmol) is added in small portion to a mixture of 1-isopropyl-5-nitro-1,3-dihydro-indol-2-one (2.60 g, 11.8 mmol) and ammonium chloride (6.27 g, 118 mmol) in ethanol (80 ml) and water (40 ml) at 90° C. The reaction mixture is stirred vigorously and heated for 45 min, then cooled to room temperature and diluted with dichloromethane (250 ml). The mixture is filtered through celite, the organic layer separated and washed with water and brine, dried (Na₂SO₄) and evaporated to provide the title compound. HPLC r.t. 2.51 min; MS for C₁₁H₁₄N₂O m/z 191.1 (+M)⁺.

Step 5. Preparation of (R)-[2-hydroxy-3-(4-isopropyl-2-oxo-2,3-dihydro-1H-indol-5-ylamino)-propyl]-carbamic Acid tert-butyl Ester.

5-Amino-1-isopropyl-1,34-dihydro-indol-2-one (0.76 g, 3.99 mmol), (S)-oxiranylmethyl-carbamic acid tert-butyl ester (0.69 g, 3.99 mmol) and lithium trifluoromethanesulfonate (0.617 g, 3.99 mmol) in acetonitrile (10 ml) is heated at 70° C. for 2 hours. The reaction is diluted with ethyl acetate, washed with water and brine, dried (Na₂SO₄) and evaporated. Final purification by flash chromatography (20% Acetone/DCM) provide the title compound. HPLC r.t. 3.61 min; MS for C₁₉H₂₉N₃O₄ m/z 364.0 (M+1)⁺.

Step 6. Preparation of (S)-[3-(1-isopropyl-2-oxo-2,3-dihydro-1H-indol-5-yl)-2-oxo-oxazolidin-5-ylmethyl]-carbamic Acid tert-butyl Ester.

Phosgene (20% solution in toluene, 0.602 ml, 6.18 mmol) is added to (5R)-[2-hydroxy-3-(1-isopropyl-2-oxo-2,3-dihydro-1H-indol-5-ylamino)-propyl]-carbamic acid tert-butyl ester (0.15 g, 0.412 mmol) and triethylamine (0.28 ml, 2.06 mmol) in dichloromethane (5 ml) at 0° C. The reaction is allowed to warm to room temperature and stirred for 2 h. The mixture is diluted with dichloromethane, washed with water and brine, dried (Na₂SO₄) and evaporated. Final purification by PTLC (5% MeOH/DCM) provides the title compound. HPLC r.t. 4.78 min; MS for C₂₀H₂₇N₃O₅ m/z 390.3 (M+H)⁺.

Step 7. Preparation of (R)-(5-aminomethyl-2-oxo-oxazolidin-3-yl)-1-isopropyl-1,3-dihydro-indole-2-one

(5R)-[3-Isopropyl-2-oxo-2,3-hydro-1H-indol-5-yl)-2-oxo-oxazolidin-5-ylmethyl]-carbamic acid tert-butyl ester (0.25 g, 0.642 mmol) is treated with 50% TFA/DCM (4 ml) for 30 minutes at room temperature. The reaction is evaporated to provide the title compound. HPLC r.t. 3.06 min; MS for C₁₅H₁₉N₃O₃ m/z 290.2 (M+H)⁺.

Step 8. Preparation of (R)-5-((1H-1,2,3-triazol-1-yl)methyl)-3-isopropyl-2-oxoindolin-5-yl)oxazolidin-2-one.

Dichloroacetaldehyde (0.10 g, 0.0009 mol), p-toluenesulfonhydrazide (0.16 g, 0.0009 mol) and acetic acid (0.02 mL, 0.0004 mol) in methanol (3 mL) are stirred at room temperature for 1 h. The resulting white suspension of N-(2,2-dichloroethylidene)-4-methylbenzenesulfonohydrazide is cooled in an ice water bath and a mixture of (S)-5-(aminomethyl)-3-(1-isopropyl-2-oxoindolin-5-yl)oxazolidin-2-one (0.3 g, 0.0009 mol) and triethylamine (0.3 mL, 0.0018 mol) in dimethylformamide (5 mL) added in one portion. The mixture is stirred at room temperature overnight and the solvent removed under reduced pressure. The residue is purified by PTLC (10% methanol/dichloromethane) to provide the title compound. HPLC(SYMMETRY C₁₈ 3.5 μM, 4.6×30 mm column; gradient elution 2%-98% MeCN with 0.1% TFA over 10 min; 2 mL/min rate): retention time=3.709 min; ¹H NMR (300 MHz, DMSO-d₆): 7.86 (d, J=2.2 Hz, 1H), 7.75 (d, J=2.2 Hz, 1H), 7.43 (s, 1H), 7.24 (d, J=8.5 Hz, 1H), 7.01 (d, J=8.5 Hz, 1H), 5.06 (m, 1H), 4.86 (m, 1H), 4.73 (d, J=5.2 Hz, 2H), 4.58 (m, 2H), 4.19 (t, J=9.1 Hz, 1H), 3.82 (dd, J=5.8, 2.7 Hz, 1H), 3.56 (s, 2H), 1.57 (d, J=6.7 Hz, 6H); MS for C₁₇H₁₉N₅O₃ (m/z) 342.1 (M+H)⁺.

### Example 2

### Preparation of (R)-5-(1H-1,2,3-triazol-1-yl)methyl)-3-(1-ethyl-2-oxoindolin-5-yl)oxazolidin-2-one

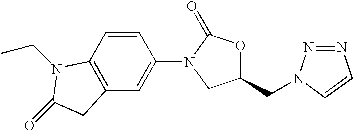

Following the general procedure of Example 1, and making non-critical variations but substituting iodoethane as starting material (5.44 ml, 0.068 mol), the title compound is obtained. HPLC (SYMMETRY C₁₈ 3.5 μM, 4.6×30 mm column; gradient elution 2%-98% MECN with 0.1% TFA over 10 min; 2 mL/min rate): retention time=3.642 min; ¹H NMR (300 MHz, DMSO-d₆): 7.87 (d, J=2.2 Hz, 1H), 7.74 (d, J=2.2 Hz, 1H), 7.43 (s, 1H), 7.24 (d, J=8.5 Hz, 1H), 7.01 (d, J=8.5 Hz, 1H), 5.06 (m, 1H), 4.73 (d, J=5.2 Hz, 2H), 4.19 (t, J=9.1 Hz, 1H), 3.82 (dd, J=5.8, 2.7 Hz, 1H), 3.60 (m, 4H), 1.18 (t, J=6.7 Hz, 3H); MS for C₁₆H₁₇N₅O₃ (m/z) 328.2 (M+H)⁺.

We claim:

1. A compound of formula I 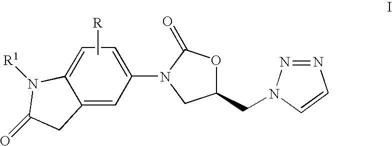
or a pharmaceutically acceptable salt thereof wherein R is H or F; and R¹ is C₁₋₄alkyl.

2. A compound of claim 1 which is
(1) (R)-5-((1H-1,2,3-triazol-1-yl)methyl)-3-(1-isopropyl-2-oxoindolin-5-yl)oxazolidin-2-one,
(2) (R)-5-((1H-1,2,3-triazol-1-yl)methyl)-3-(1-ethyl-2-oxoindolin-5-yl)oxazolidin-2-one, or
(3) (R)-5-((1H-1,2,3-triazol-1-yl)methyl)-3-(1-methyl-2-oxoindolin-5-yl)oxazolidin-2-one.

3. A pharmaceutical composition comprising a compound of claim 1 or a pharmaceutically acceptable salt thereof, and a pharmaceutically acceptable carrier.

4. A method for treating bacteria infections comprising administering to a mammal being treated a pharmaceutically effective amount of the compound of claim 1.

5. The method of claim 4 wherein the compound of claim 1 is administered orally, parenterally, topically, rectally, or intranasally.

6. The method of claim 4 wherein said compound is administered in an amount of from about 0.1 to about 100 mg/kg of body weight/day.

7. The method of claim 4 wherein said compound is administered in an amount of from about 1 to about 50 mg/kg of body weight/day.

8. The bacteria infection of claim 4 which is ear infections, eye infections, respiratory tract infections, skin and skin structure infections, bacterial endocarditis, osteomyelitis, endocarditis or diabetic foot.

9. The bacteria infection of claim 4 which is caused by gram-positive bacteria, gram negative bacteria, anaerobic organisms, and acid-fast organisms.

10. The bacteria infection of claim 4 which is caused by bacteria comprising staphylococci, streptococci, Enterococci, Haemophilus, Moraxella, bacteroides, clostridia, Mycobacteria, or Chlamydia.

11. The bacteria of claim 10 wherein staphylococci is S. aureus and S. epidermidis; wherein streptococci is S. pneumoniae of S. pyogenes; wherein Enterococci is E. faecalis; wherein Haemophilus is H. influenzae; wherein Moraxella is M. catarrhalis; and wherein Mycobacteria is M. tuberculosis; or Mycobacterium avium.

12. The bacteria infections of claim 4 which is caused by multi-drug resistant S. aureus.
